(19) United States
(12) Patent Application Publication
(10) Pub. No.: US 2026/0201376 A1
(43) Pub. Date: Jul. 16, 2026

(54) ANGIOGENIC MICRORNAS

(71) Applicant: SUMMA BIOTECH B.V., Utrecht (NL)

(72) Inventors: Despoina KESIDOU, EDINBURGH (GB); Abdelaziz BEQQALI, EDINBURGH (GB); Andrew BAKER, EDINBURGH (GB)

(21) Appl. No.: 19/136,253

(22) PCT Filed: Dec. 8, 2023
(86) PCT No.: PCT/EP2023/084938

§ 371 Date: Jun. 5, 2025

(30) Foreign Application Priority Data
Dec. 9, 2022 (EP) 22386090.9

(51) Int. Cl. C12N 15/113; A61P 9/00

(52) U.S. Cl. CPC C12N 15/113; A61P 9/00; C12N 2310/141

(57) ABSTRACT

The invention relates to compositions comprising a microRNA having a pro-angiogenic effect. In particular, the invention relates to a composition comprising a microRNA selected from hsa-miR-4691-5p, hsa-miR-4496, hsa-miR-582-5p, and hsa-miR-345-3p. The invention further relates to nucleic acid molecules encoding at least one of these microRNAs and viral particles capable of expressing these microRNAs in a cell. The invention further relates to the use of these microRNAs, nucleic acid molecules or viral particles as medicine, particularly in the treatment of a cardiovascular disease.

### FIELD OF THE INVENTION

The invention relates to compositions comprising a microRNA having a pro-angiogenic effect. In particular, the invention relates to a composition for use for use in the treatment, prevention or amelioration of a disease, wherein the disease is a cardiovascular disease, and wherein the use comprises inducing neovascularization, the composition comprising a microRNA selected from hsa-miR-4691-5p, hsa-miR-4496, hsa-miR-582-5p, and hsa-miR-345-3p or variants thereof.

### BACKGROUND OF THE INVENTION

Cardiovascular disease (CVD) is the most common cause of mortality worldwide with the World Health Organisation (WHO) reporting that in 2017, 17.9 million people died from CVDs. Annual costs of CVD to the NHS and wider society are estimated to be £15.8 per year. The most common CVD is coronary heart disease (CHD) characterised by the narrowing of the coronary arteries which may lead to ischaemic damage and cardiomyocyte death, a phenomenon known as myocardial infarction (MI).

Current treatment includes interventional procedures (balloon angioplasty usually combined with stent insertion) or surgery to open up or bypass blocked arteries. Nonetheless, several limitations relate to these treatments including allergic reactions to the inserted materials, arterial damage, re-narrowing of the arteries and blood clots.

Therefore there is an ever existing need for new treatment options to address coronary heart disease. The invention as defined in the appended claims aims to address this ongoing need.

### FIGURE LEGENDS

FIG. 1. Negligible expression of most novel miRNAs with a potential role in angiogenesis in EC and SMC panels. A. Expression of hsa-miRNA-4496 in HCMECs, HSVECs, HCAECs, HPAECs, HSVSMCs, HCASMCs, HPASMCs. The graphs depict the ΔCt values (left) to housekeeper gene (RNU) and Ct values (right) of the miRNA and the housekeeper gene (RNU). B. Expression of hsa-miRNA-4691-5p in HCMECs, HSVECs, HCAECs, HPAECs, HSVSMCs, HCASMCs, HPASMCs. The graph depicts the Ct values of the miRNA and the housekeeper gene (RNU). Since the expression of this miRNA was undetectable in the cell panel tested ΔCt values were not plotted. C. Expression of hsa-miRNA-582-5p in HCMECs, HSVECs, HCAECs, HPAECs, HSVSMCs, HCASMCs, HPASMCs. The graphs depict the ΔCt values (left) to housekeeper gene (RNU) and Ct values (right) of the miRNA and the housekeeper gene (RNU). D. Expression of hsa-miRNA-345-3p in HCMECs, HSVECs, HCAECs, HPAECs, HSVSMCs, HCASMCs, HPASMCs. The graphs depict the ΔCt values (left) to housekeeper gene (RNU) and Ct values (right) of the miRNA and the housekeeper gene (RNU). E. Expression of hsa-miRNA-579-3p in HCMECs, HSVECs, HCAECs, HPAECs, HSVSMCs, HCASMCs, HPASMCs. The graph depicts the Ct values of the miRNA and the housekeeper gene (RNU). Since the expression of this miRNA was undetectable in the cell panel tested ΔCt values were not plotted. F. Expression of hsa-miRNA-3692-3p in HCMECs, HSVECs, HCAECs, HPAECs, HSVSMCs, HCASMCs, HPASMCs. The graph depicts the Ct values of the miRNA and the housekeeper gene (RNU). Since the expression of this miRNA was undetectable in the cell panel tested ΔCt values were not plotted.

FIG. 2. Overexpression of miR-345-3p, or miR-582-5p, or miR-4496 or miR-4691-5p results in increased EC tube formation and migration. A. qRT-PCR showing the expression of the 6 miRNA candidates post overexpression with miRNA mimics (n=3) B. Tube Formation Assay on HCMECs transfected with the 6 miRNA candidates (miR-345-3p, miR-579-3p, miR-582-5p, miR-3692-3pk,kk, miR-4496 or miR-4691-5p) (n=3). Cells transfected with miR-126-5p served as positive control. Cells transfected with a miRNA control (miRC) served as the negative control. Staining with Calcein AM confirmed cell viability in all conditions. Experiments were performed both in basal conditions (GF−) and in fully supplemented media (GF+). Data were analysed using the “Angiogenesis Analyzer” tool. Scale bars=100 μm C. Migration Assay on HCMECs transfected with the 6 miRNA candidates (miR-345-3p, miR-579-3p, miR-582-5p, miR-3692-3p, miR-4496 or miR-4691-5p) (n=3). Cells transfected with miR-126-5p served as positive control. Cells transfected with a miRC served as the negative control. Experiments were performed both in basal conditions (GF−) and in fully supplemented media (GF+). Data were analysed using the “MRI Wound Healing” Tool. Scale bars=100 μm. Statistical significance was determined by one-way ANOVA with Dunnett's multiple comparisons test. Error bars represent the SD. D. EdU proliferation assay on HCMECs transfected with the 6 miRNA candidates (miR-345-3p, miR-579-3p, miR-582-5p, miR-3692-3p, miR-4496 or miR-4691-5p) (n=3). Cells transfected with miR-126-5p served as positive control. Cells transfected with a miRC served as the negative control. Experiments were performed both in basal conditions (GF−) and in fully supplemented media (GF+). Statistical significance was determined by one-way ANOVA with Dunnett's multiple comparisons test. Error bars represent the SD.

FIG. 3. Principal Component Analysis (PCA) of HCMECs overexpressing the 4 novel miRNAs showed a clear separation to miRC-transfected samples. The batch effect of the different HCMEC biological replicates was informatically removed using the R package limma

FIG. 4. Volcano plots representing differential expression analyses between samples overexpressing the 4 novel miRNAs (miR-345-3p, miR-582-5p, miR-4496, or miR-4691-5p) and samples transfected with a miRNA control (miRC). Data are shown as −log₁₀P (y-axis) to log₂FC log₂ fold change (log₂FC) (x-axis). Differentially expressed genes (log₂FC>1.5) are labelled in green, genes with a p-value<0.05 are labelled in blue, and significantly differentially expressed genes (log₂FC>1.5 and p-value<0.05) are labelled in red. Non-significantly (NS) differentially expressed genes are labelled in grey.

FIG. 5. Overexpression of the 4 novel miRNAs results in distinct transcriptomic signatures compared to the miRC. Heatmaps representing differentially expressed miRNAs among miR-345-3p, miR-582-5p, miR-4496 or miR-4691-5p overexpressing and miRC transfected HCMECs as Z-score of log2 (DESeq normalized counts+1), Ifc>1.5, p<0.05

FIG. 6. The 4 novel miRNAs control genes involved in the regulation of cell cycle. GO terms of upregulated genes post miRNA overexpression. Genes with FPKM<1 in the miRNA-overexpressing or miRC transfected samples were filtered out of the analysis. All genes with FPKM>1 in the miRNA-overexpressing or miRC transfected samples were used as the background for this analysis.

### SUMMARY OF THE INVENTION

In a first aspect the invention relates to a composition for use for use in the treatment, prevention or amelioration of a disease,
wherein the disease is a cardiovascular disease, and wherein the use comprises inducing neovascularization; and
wherein the composition comprises one or more microRNAs selected from the group consisting of:

hsa-miR-4691-5p
(guccuccaggccaugagcugcgg),
hsa-miR-4496
(gaggaaacugaagcugagaggg),
hsa-miR-582-5p
(uuacaguuguucaaccaguuacu),
and
hsa-miR-345-3p
(gcccugaacgaggggucuggag),
or a variants thereof.

### Definitions

A portion of this disclosure contains material that is subject to copyright protection (such as, but not limited to, diagrams, device photographs, or any other aspects of this submission for which copyright protection is or may be available in any jurisdiction). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein. “A,” “an,” and “the”: these singular form terms include plural referents unless the content clearly dictates otherwise. Thus, for example, reference to “a cell” includes a combination of two or more cells, and the like.

“About” and “approximately”: these terms, when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Agonist”: This term, as used herein refers to a compound or agent having the ability to initiate or enhance a biological function of a target protein or polypeptide, such as increasing the activity or expression of the target protein or polypeptide. Accordingly, the term “agonist” is defined in the context of the biological role of the target protein or polypeptide. While some agonists herein specifically interact with (e.g., bind to) the target, compounds and/or agents that initiate or enhance a biological activity of the target protein or polypeptide by interacting with other members of the signal transduction pathway of which the target polypeptide is a member are also specifically included within this definition.

“Antagonist” and “inhibitor”: These terms are used interchangeably, and they refer to a compound or agent having the ability to reduce or inhibit a biological function of a target protein or polypeptide, such as by reducing or inhibiting the activity or expression of the target protein or polypeptide. Accordingly, the terms “antagonist” and “inhibitor” are defined in the context of the biological role of the target protein or polypeptide. An inhibitor need not completely abrogate the biological function of a target protein or polypeptide, and in some embodiments reduces the activity by at least 20%, 30%, 40%, 50%, 60%, 70%, 80%, 90%, 95%, or 99%. While some antagonists herein specifically interact with (e.g., bind to) the target, compounds that inhibit a biological activity of the target protein or polypeptide by interacting with other members of the signal transduction pathway of which the target protein or polypeptide are also specifically included within this definition. Non-limiting examples of biological activity inhibited by an antagonist include those associated with the development, growth, or spread of a tumor, or an undesired immune response as manifested in autoimmune disease.

“Anti-cancer effect”: This refers to the effect a therapeutic agent has on cancer, e.g., a decrease in growth, viability, or both of a cancer cell. The IC50 of cancer cells can be used as a measure the anti-cancer effect. IC50 refers to a measure of the effectiveness of a therapeutic agent in inhibiting cancer cells by 50%. “Alleviating cancer”: The term, in the context of specific cancers and/or their pathologies, refers to degrading a tumor, for example, breaking down the structural integrity or connective tissue of a tumor, such that the tumor size is reduced when compared to the tumor size before treatment. “Alleviating” metastasis of cancer includes reducing the rate at which the cancer spreads to other organs.

“And/or”: The term “and/or” refers to a situation wherein one or more of the stated cases may occur, alone or in combination with at least one of the stated cases, up to with all of the stated cases.

“Cancer”: This term refers to the physiological condition in mammals that is typically characterized by unregulated cell growth. The terms “cancer,” “neoplasm,” and “tumor,” are often used interchangeably to describe cells that have undergone a malignant transformation that makes them pathological to the host organism. Primary cancer cells can be distinguished from non-cancerous cells by techniques known to the skilled person. A cancer cell, as used herein, includes not only primary cancer cells, but also cancer cells derived from such primary cancer cell, including metastasized cancer cells, and cell lines derived from cancer cells. Examples include solid tumors and non-solid tumors or blood tumors. Examples of cancers include, without limitation, leukemia, lymphoma, sarcomas and carcinomas (e.g. colon cancer, pancreatic cancer, breast cancer, ovarian cancer, prostate cancer, lung cancer, melanoma, lymphoma, non-Hodgkin lymphoma, colon cancer, (malignant) melanoma, thyroid cancer, papillary thyroid carcinoma, lung cancer, non-small cell lung carcinoma, and adenocarcinoma of lung). As is well known, tumors may metastasize from a first locus to one or more other body tissues or sites. Reference to treatment for a “neoplasm, “tumors” or “cancer” in a patient includes treatment of the primary cancer, and, where appropriate, treatment of metastases.

“Compositions”, “products” or “combinations”: These encompass those compositions suitable for various routes of administration, including, but not limited to, intravenous, subcutaneous, intradermal, subdermal, intranodal, intratumoral, intramuscular, intraperitoneal, oral, nasal, topical (including buccal and sublingual), rectal, vaginal, aerosol and/or parenteral or mucosal application. The compositions, formulations, and products according to the disclosure invention normally comprise the drugs/compound/inhibitor (alone or in combination) and one or more suitable pharmaceutically acceptable excipients or carriers.

“Conventional techniques” or “methods known to the skilled person”: These terms refer to a situation wherein the methods of carrying out the conventional techniques used in methods of the invention will be evident to the skilled worker. The practice of conventional techniques in molecular biology, biochemistry, cell culture, genomics, sequencing, medical treatment, pharmacology and related fields are well-known to those of skill in the art and are discussed, for example, in the following literature references: Human Embryonic Stem Cell: The Practical Handbook. Publisher: John Wiley & Sons, LTD, Editors (Sullivan, S., Cowan, C. A., Eggan, K.) Harvard University, Cambridge, MA, USA (2007); Human Stem Cell, a Laboratory Guide (2nd Edition) by Peterson, S., and Loring, J. F. (2012).

“Comprising”: this term is construed as being inclusive and open ended, and not exclusive. Specifically, the term and variations thereof mean the specified features, steps or components are included. These terms are not to be interpreted to exclude the presence of other features, steps or components.

“Exemplary”: this terms means “serving as an example, instance, or illustration,” and should not be construed as excluding other configurations disclosed herein.

As used herein, “Percent (%) nucleic acid sequence identity” with respect to a reference polynucleotide sequence is defined as the percentage of nucleic acid residues in a candidate sequence that are identical with the nucleic acid residues in the reference polynucleotide sequence, after aligning the sequences and introducing gaps, if necessary, to achieve the maximum percent sequence identity, and not considering any conservative substitutions as part of the sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % nucleic acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for nucleic acid sequence comparisons, the % nucleic acid sequence identity of a given nucleic acid sequence A to, with, or against a given nucleic acid sequence B (which can alternatively be phrased as a given nucleic acid sequence A that has or comprises a certain % nucleic acid sequence identity to, with, or against a given nucleic acid sequence B) is calculated as follows:

100 times the fraction X/Y

where X is the number of nucleic acid residues scored as identical matches by the sequence alignment program ALIGN-2 in that program's alignment of A and B, and where Y is the total number of nucleic acid residues in B. It will be appreciated that where the length of nucleic acid sequence A is not equal to the length of nucleic acid sequence B, the % nucleic acid sequence identity of A to B will not equal the % nucleic acid sequence identity of B to A. Unless specifically stated otherwise, all % nucleic acid sequence identity values used herein are obtained as described in the immediately preceding paragraph using the ALIGN-2 computer program.

As used herein the term “nucleic acid” or “polynucleotide” refers to any polymers or oligomers of (contiguous) nucleotides. The nucleic acid may be DNA or RNA, or a mixture thereof, and may exist permanently or transitionally in single-stranded or double-stranded form, including homoduplex, heteroduplex, and hybrid states. The present invention contemplates any deoxyribonucleotide, ribonucleotide or peptide nucleic acid component, and any chemical variants thereof, such as methylated, hydroxymethylated or glycosylated forms of these bases, and the like. The polymers or oligomers may be heterogeneous or homogenous in composition, and may be isolated from naturally occurring sources or may be artificially or synthetically produced. The term “isolated” thus means isolated from naturally occurring sources or artificially or synthetically produced.

### DETAILED DESCRIPTION OF THE INVENTION

It is contemplated that any method, use or composition described herein can be implemented with respect to any other method, use or composition described herein. Embodiments discussed in the context of methods, use and/or compositions of the invention may be employed with respect to any other method, use or composition described herein. Thus, an embodiment pertaining to one method, use or composition may be applied to other methods, uses and compositions of the invention as well. Current treatment of coronary heart disease includes interventional procedures (balloon angioplasty usually combined with stent insertion) or surgery to open up or bypass blocked arteries. Nonetheless, several limitations relate to these treatments including allergic reactions to the inserted materials, arterial damage, re-narrowing of the arteries and blood clots. For this reason, therapeutic neovascularisation has been proposed as a possible alternative strategy to create new myocardial blood vessel networks above the infarct site and reduce the extent of cardiomyocyte damage.

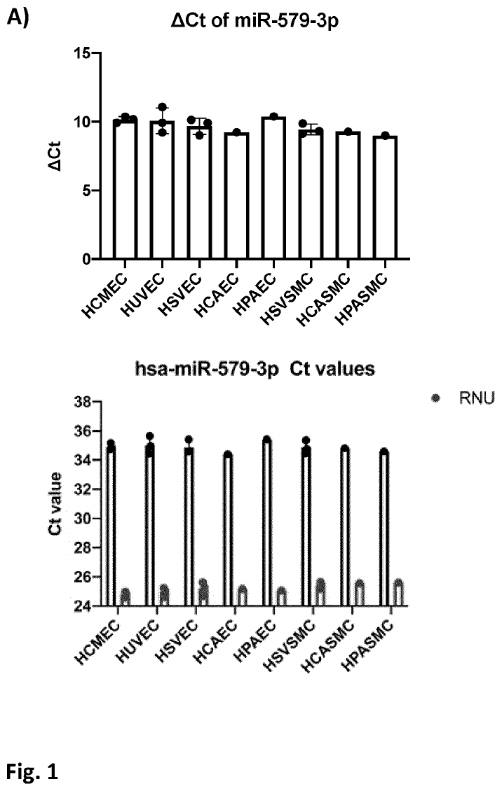

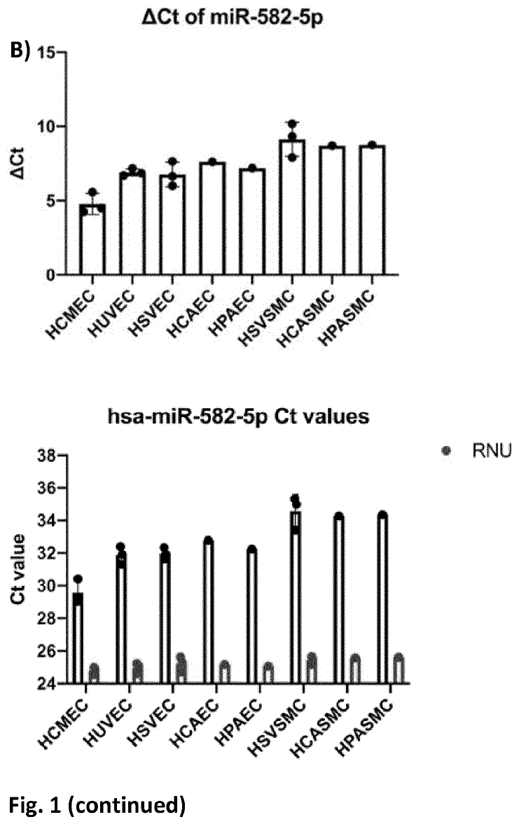

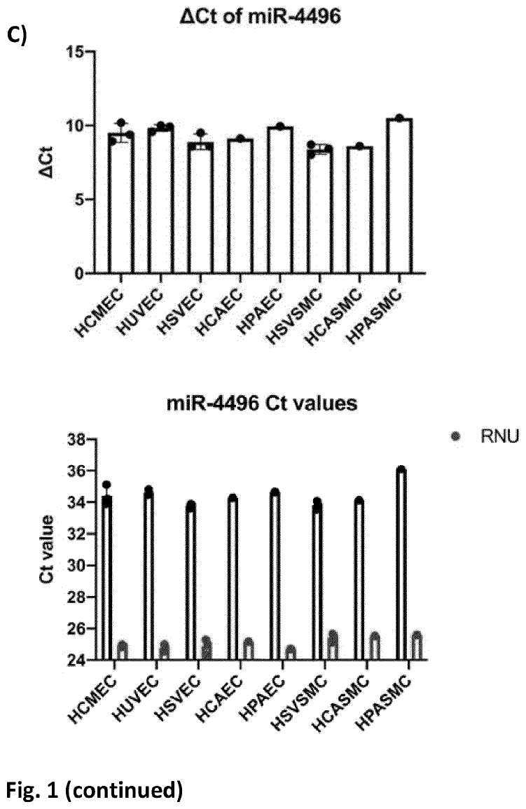

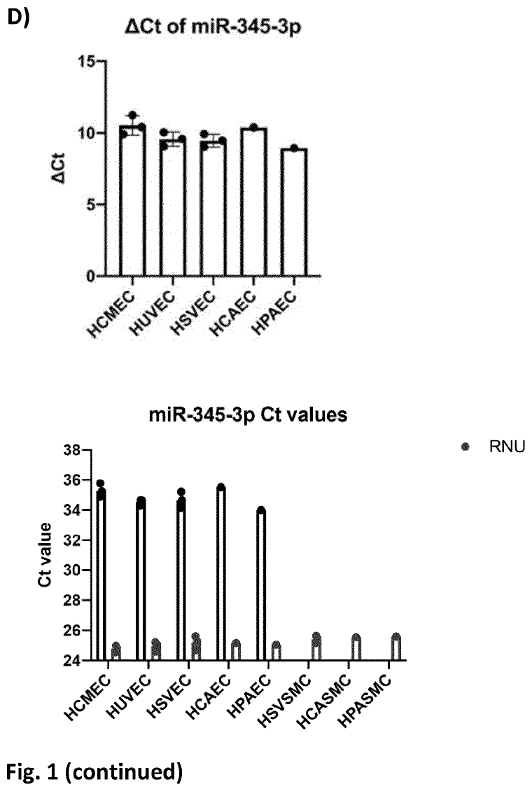

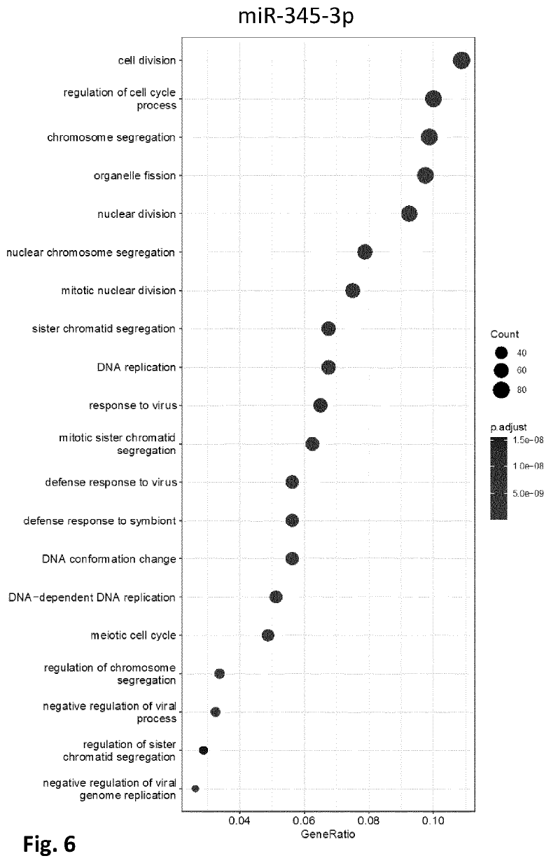

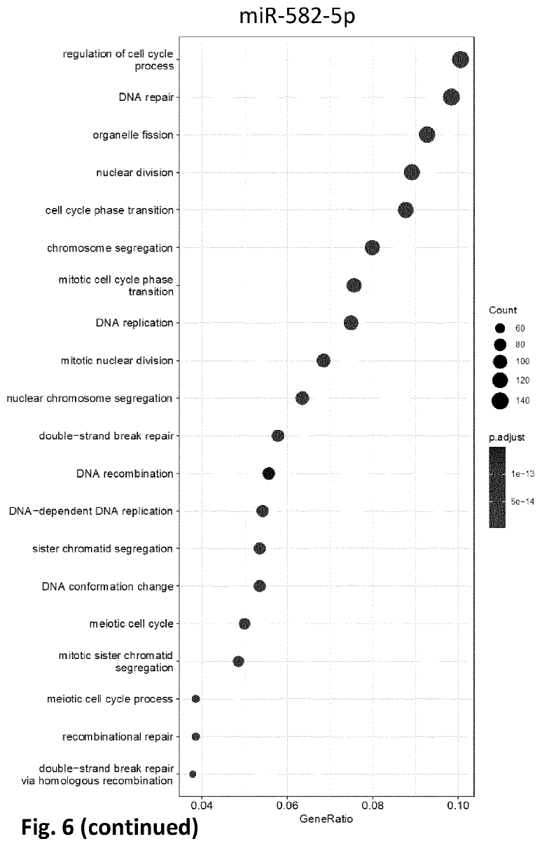

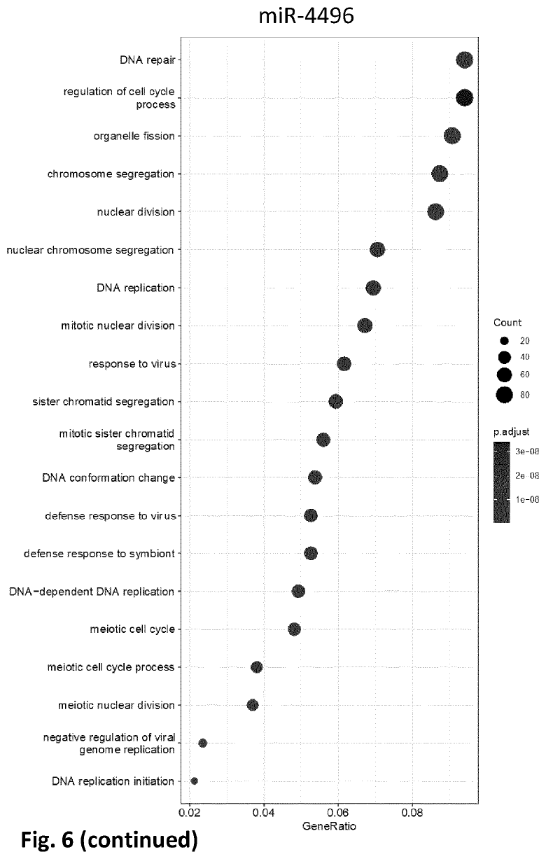

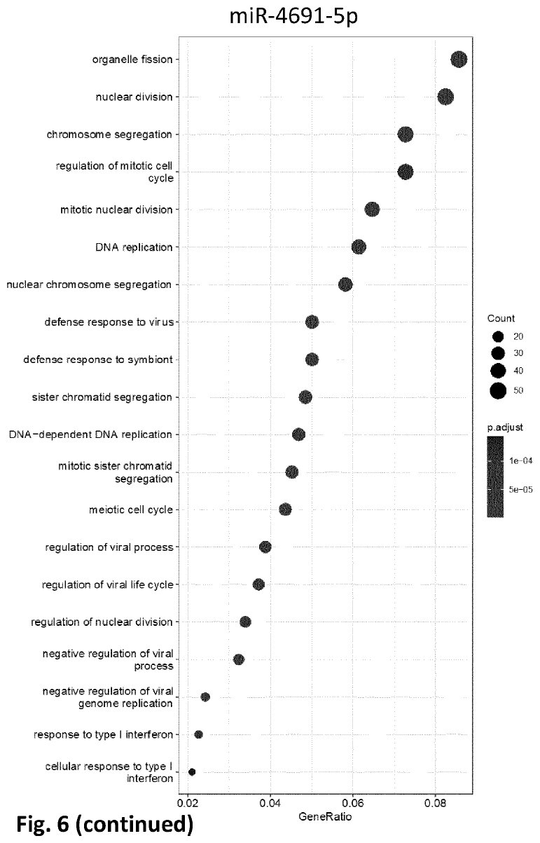

MiRNAs have emerged as promising candidates for therapeutic neovascularisation. Aiming to identify novel miRNAs with a potential role in angiogenesis, the inventors performed a bioinformatics analysis investigating the implication of miRNAs with angiogenesis pathways. In this analysis, the inventors identified 14 novel miRNAs with a potential role in angiogenesis and selected 6 miRNA candidates for experimental validation. The inventors demonstrated that 4 out of the 6 novel miRNAs (hsa-miR-345-3p, hsa-miR-582-5p, hsa-miR-4496, hsa-miR-4691-5p) effectively induced EC angiogenesis and by deep RNA sequencing showed that the 4 novel angiogenic miRNAs control genes related to the regulation of cell cycle, nuclear division and DNA repair. The absence/minimal expression of these candidates in endothelial cells (ECs) as well as their ability to get sorted in extracellular vesicles (EVs) offers a great advantage for their clinical application for therapeutic neovascularisation. These results are described in Example 1 and corresponding FIGS. 1-6.

Therefore, disclosed herein is a composition comprising at least one microRNA selected from the group consisting of hsa-miR-4691-5p (guccuccaggccaugagcugcgg), hsa-miR-4496 (gaggaaacugaagcugagaggg), hsa-miR-582-5p (uuacaguuguucaaccaguuacu), and hsa-miR-345-3p (gcccugaacgaggggucuggag) or a variant thereof. Further disclosed is a composition comprising a nucleotide encoding one or more microRNAs as defined herein. Further disclosed is viral particle comprising the microRNA as defined herein or a nucleotide encoding a microRNA as defined herein. Further disclosed herein is the microRNA, nucleotide or viral particle for use as a medicament. In an embodiment the composition is a pharmaceutical composition.

In a first aspect, the invention relates to a composition for use for use in the treatment, prevention or amelioration of a disease, wherein the disease is a cardiovascular disease, and wherein the use comprises inducing neovascularization; and wherein the composition comprises one or more microRNAs selected from the group consisting of hsa-miR-4691-5p (guccuccaggccaugagcugcgg, SEQ ID NO: 1), hsa-miR-4496 (gaggaaacugaagcugagaggg, SEQ ID NO: 2), hsa-miR-582-5p (uuacaguuguucaaccaguuacu, SEQ ID NO: 3), and hsa-miR-345-3p (gcccugaacgaggggucuggag, SEQ ID NO: 4) or a variants thereof.

The sequences of the four microRNAs are described with SEQ ID Nos: 1-4. Therefore in an embodiment the invention relates to a pharmaceutical composition comprising at least one microRNA comprising or consisting of a sequence selected from the group consisting of SEQ ID NO: 1, SEQ ID NO: 2, SEQ ID NO: 3, or SEQ ID NO: 4 or a variant thereof.

The inventors have demonstrated that microRNAs hsa-miR-4691-5p, hsa-miR-4496, hsa-miR-582-5p, and hsa-miR-345-3p have proangiogenic effect. This effect is demonstrated using tube formation assays and HCMEC migration and proliferation assays (see Examples). It is understood that microRNAs with similar but not identical sequence may be used to achieve the same pro-angiogenic effect, therefore the invention also intend to cover variants of these four microRNAs.

Therefore in an embodiment the variant comprises or consist of a nucleotide sequence with a sequence identity of at least 70%, e.g. 70%, 75%, 80%, 82%, 84%, 86%, 88%, 90%, 91%, 92%, 93%, 94%, 95%, 96%, 97%, 98%, 99%, or 100%, to hsa-miR-4691-5p (guccuccaggccaugagcugcgg), hsa-miR-4496 (gaggaaacugaagcugagaggg), hsa-miR-582-5p (uuacaguuguucaaccaguuacu), and hsa-miR-345-3p (gcccugaacgaggggucuggag). Thus the invention also relates to a pharmaceutical composition comprising at least one microRNA comprising or consisting of a sequence which has at least 70%, e.g. 70%, 75%, 80%, 82%, 84%, 86%, 88%, 90%, 91%, 92%, 93%, 94%, 95%, 96%, 97%, 98%, 99%, or 100%, sequence homology with SEQ ID NO: 1, SEQ ID NO: 2, SEQ ID NO: 3 or SEQ ID NO: 4.

In an embodiment the variant comprises or consists of a nucleotide sequence containing up to 8 altered nucleotides in hsa-miR-4691-5p (guccuccaggccaugagcugcgg), hsa-miR-4496 (gaggaaacugaagcugagaggg), hsa-miR-582-5p (uuacaguuguucaaccaguuacu), and hsa-miR-345-3p (gcccugaacgaggggucuggag). Thus the invention also relates to a pharmaceutical composition comprising at least one microRNA comprising or consisting of a sequence which has up to 8 altered nucleotides with respect to SEQ ID NO: 1, SEQ ID NO: 2, SEQ ID NO: 3 or SEQ ID NO: 4.

It is understood that the seed sequence is of importance for the effect of a microRNA to downregulate its target sequence. The seed sequence of a miRNA is defined as the first 2-8 nucleotides starting at the 5′ end and counting toward the 3′ end. Therefore the variant microRNA preferably has a seed sequence which is identical or contains up to 1 altered nucleotides with respect to the seed sequences as defined in SEQ ID NO: 1, SEQ ID NO: 2, SEQ ID NO: 3 or SEQ ID NO: 4, being nucleotides 2-8 starting from the 5′ end of the respective sequences.

It is further understood that G-U wobble base pairs may be formed between the microRNA and its target. The G-U wobble base pair is a fundamental unit of RNA secondary structure that is present in nearly every class of RNA from organisms of all three phylogenetic domains. It has comparable thermodynamic stability to Watson-Crick base pairs and is nearly isomorphic to them. Therefore for the purpose determining sequence complementarity a G-U wobble pair is not considered a mismatch. Thus where an “A” in the sequence is replaced by a “G” or a “C” is replaced by an “U” the resulting bases can still pair to from a G-U wobble and should be counted as having sequence homology. In an embodiment the seed sequence comprises a maximum of two, preferably maximum one G-U wobble base pairs.

In an embodiment that microRNAs or variants thereof induce neovascularization when administered to a subject or introduced in a cell, preferably an endothelial cell.

The microRNA hsa-mir-4691 is described by accession number MI0017324 in miRbase or Ensembl entry ENSG00000284435 and may further be referred to as MIR4691, which is the full length hairpin also listed herein as SEQ ID NO: 5. From the hairpin the mature sequence hsa-miR-4691-5p is derived, which is described by accession number MIMAT0019781 in miRbase and described herein as SEQ ID NO: 1.

The microRNA hsa-mir-4496 is described by accession number MI0016858 miRbase or Ensembl entry ENSG00000284388 and may further be referred to as MIR4496, which is the full length hairpin also listed herein as SEQ ID NO: 6. From the hairpin the mature sequence hsa-miR-4496 is derived, which is described by accession number MIMAT0019031 in miRbase and described herein as SEQ ID NO: 2.

The microRNA hsa-mir-582 is described by accession number MI0003589 in miRbase or Ensembl entry ENSG00000202601 and may further be referred to as MIR582, which is the full length hairpin also listed herein as SEQ ID NO: 7. From the hairpin the mature sequence hsa-miR-582-5p is derived, which is described by accession number MIMAT0003247 in miRbase and described herein as SEQ ID NO: 3.

The microRNA hsa-mir-345 is described by accession number MI0000825 in miRbase or Ensembl entry ENSG00000198984 and may further be referred to as MIR345, which is the full length hairpin also listed herein as SEQ ID NO: 8. From the hairpin the mature sequence hsa-miR-345-3p is derived, which is described by accession number MIMAT0022698 in miRbase and described herein as SEQ ID NO: 4.

Therefore, in an embodiment the at least one microRNA as described herein comprises or consists of a sequence selected from the group consisting of SEQ ID NO: 5, SEQ ID NO: 6, SEQ ID NO: 7, or SEQ ID NO: 8, or a variant thereof, the variant being defined as a microRNA comprising or consisting of a sequence which has at least 70%, e.g. 70%, 75%, 80%, 82%, 84%, 86%, 88%, 90%, 91%, 92%, 93%, 94%, 95%, 96%, 97%, 98%, 99%, or 100%, sequence homology with SEQ ID NO: 5, SEQ ID NO: 6, SEQ ID NO: 7 or SEQ ID NO: 8.

As the microRNAs identified by the inventors have a proangiogenic effect, the variants intended to be covered by the invention should also have a pro-angiogenic effect. Therefore in an embodiment the microRNA as broadly defined herein has a pro-angiogenic effect when expressed in an endothelial cell. Methods to express the microRNAs or variants thereof in a endothelial cell are known to the skilled person, and may for example be performed transiently by transfection of the cell using an appropriate transfecting agent or by introducing an expression vector in the cell capable of expressing the microRNA (or variant thereof). Methods to determine an angiogenic effect of a variant are readily available and may for example comprise tube formation assays or wound healing assays as described in the examples below. Suitable cell lines for performing such tests are for example but not limited to Human primary aortic endothelial cells (HAEC), Human umbilical vein endothelial cells (HUVEC), human dermal microvascular endothelial cells (HMVEC), Human primary coronary artery endothelial cells (HCAEC), Human neonatal primary dermal microvascular endothelial cells (HDMVECn), Human primary pulmonary artery endothelial cells (HPAEC), human cardiac microvascular endothelial cells (HCMEC), Human saphenous vein endothelial cells (HSVEC), Human seminal vesicle smooth muscle cells (HSVSMC), Human coronary artery smooth muscle cells (HCASMC), Human pulmonary artery smooth muscle cells (HPASMC).

Therefore it is within the means of the person skilled in the art to determine whether a variant has a proangiogenic effect when expressed in an endothelial cell line without undue burden.

The inventors have identified the four microRNAs described herein based on a datamining approach. This approach is partly based on identifying microRNAs which may target components of angiogenic pathways, such as the vascular endothelial growth factor receptor (VEGFR) pathway. Putative targets for each of the microRNAs are listed below in Table 1 of the Examples. Without wishing to be bound to theory, it is anticipated that the four microRNAs exert their pro-angiogenic effect by downregulating at least one of the proteins listed in Table 1 as the putative targets of the respective microRNAs. Thus in an embodiment microRNA hsa-miR-4691-5p (guccuccaggccaugagcugcgg) or a variant thereof may target the expression of at least one protein selected from EIF2AK3 and PRKCB. In an embodiment microRNA hsa-miR-4496 (gaggaaacugaagcugagaggg) or a variant thereof may target the expression of at least one protein selected from CTNNB1, HDAC4, HDAC9, KL, NCK1, RAP1B, and ROCK2. In an embodiment microRNA hsa-miR-582-5p (uuacaguuguucaaccaguuacu) or a variant thereof may target the expression of at least one protein selected from BMP10, EIF4E, FOXO1, GSK3B, MAPK14, and RICTOR. In an embodiment microRNA hsa-miR-345-3p (gcccugaacgaggggucuggag) or a variant thereof may target the expression of at least one protein selected from CRK, GPC1, ITCH, MKNK1, PAK2, and PRKCI.

It is further envisioned that multiple microRNAs selected from miR-4691-5p, hsa-miR-4496, hsa-miR-582-5p, and hsa-miR-345-3p may be used to further increase the pro-angiogenic effect. Therefore, in an embodiment the composition as broadly described herein comprises two or more, preferably three, more preferably all four, microRNAs selected from the group consisting of hsa-miR-4691-5p (guccuccaggccaugagcugcgg), hsa-miR-4496 (gaggaaacugaagcugagaggg), hsa-miR-582-5p (uuacaguuguucaaccaguuacu), hsa-miR-345-3p (gcccugaacgaggggucuggag) or variants thereof. For example the composition may comprise hsa-miR-4691-5p and hsa-miR-4496, or hsa-miR-4691-5p and hsa-miR-582-5p, or hsa-miR-4691-5p and hsa-miR-345-3p, or hsa-miR-4496 and hsa-miR-582-5p, or hsa-miR-4496 and hsa-miR-345-3p, or hsa-miR-582-5p and hsa-miR-345-3p or variants thereof. Alternatively the composition comprises hsa-miR-4691-5p, hsa-miR-4496 and hsa-miR-582-5p, or comprises hsa-miR-4691-5p, hsa-miR-4496 and hsa-miR-345-3p, or comprises hsa-miR-4691-5p, hsa-miR-582-5p and hsa-miR-345-3p, or comprises hsa-miR-4496, hsa-miR-582-5p, and hsa-miR-345-3p or variants thereof. In an embodiment the composition comprises each of hsa-miR-4496, hsa-miR-582-5p, and hsa-miR-345-3p or variants thereof.

In an embodiment the invention relates to a pharmaceutical composition comprising at least two microRNAs comprising or consisting of sequences selected from the group consisting of SEQ ID NO: 1, SEQ ID NO: 2, SEQ ID NO: 3, or SEQ ID NO: 4 or a variant thereof. For example the composition comprises microRNAs comprising or consisting of sequences SEQ ID NO: 1 and SEQ ID NO: 2, or SEQ ID NO: 1 and SEQ ID NO: 3, or SEQ ID NO: 1 and SEQ ID NO: 4, or the composition comprises microRNAs comprising or consisting of sequences SEQ ID NO: 2 and SEQ ID NO: 3, or SEQ ID NO: 2 and SEQ ID NO: 4, or SEQ ID NO: 3 and SEQ ID NO: 4. Alternatively the composition comprises microRNAs comprising or consisting of sequences SEQ ID NO: 1, SEQ ID NO: 2 and SEQ ID NO: 3, or SEQ ID NO: 1, SEQ ID NO: 2, and SEQ ID NO: 4, or SEQ ID NO: 1, SEQ ID NO: 3 and SEQ ID NO: 4, or SEQ ID NO: 2, SEQ ID NO: 3 and SEQ ID NO: 4. Alternatively the composition comprises microRNAs comprising or consisting of each of sequences SEQ ID NO: 1, SEQ ID NO: 2, SEQ ID NO: 3 and SEQ ID NO: 4.

In an embodiment the invention relates to a pharmaceutical composition comprising at least two microRNAs comprising or consisting of sequences selected from the group consisting of SEQ ID NO: 5, SEQ ID NO: 6, SEQ ID NO: 7, or SEQ ID NO: 8 or a variant thereof. For example the composition comprises microRNAs comprising or consisting of sequences SEQ ID NO: 5 and SEQ ID NO: 6, or SEQ ID NO: 5 and SEQ ID NO: 7, or SEQ ID NO: 5 and SEQ ID NO: 8, or the composition comprises microRNAs comprising or consisting of sequences SEQ ID NO: 6 and SEQ ID NO: 7, or SEQ ID NO: 6 and SEQ ID NO: 8, or SEQ ID NO: 7 and SEQ ID NO: 8. Alternatively the composition comprises microRNAs comprising or consisting of sequences SEQ ID NO: 5, SEQ ID NO: 6 and SEQ ID NO: 7, or SEQ ID NO: 5, SEQ ID NO: 6, and SEQ ID NO: 8, or SEQ ID NO: 5, SEQ ID NO: 7 and SEQ ID NO: 8, or SEQ ID NO: 6, SEQ ID NO: 7 and SEQ ID NO: 8. Alternatively the composition comprises microRNAs comprising or consisting of each of sequences SEQ ID NO: 5, SEQ ID NO: 6, SEQ ID NO: 7 and SEQ ID NO: 8.

Herein the term pharmaceutical composition is used to refer to a composition such as for example a solution comprising the one or more microRNAs or a nucleotide as describe below, which can be used for administering to a patient or is suitable for the preparation of a medicament. The term is also intended to cover purified and lyophilized microRNAs, variants thereof or nucleotide which can be reconstituted for use in for example preparing a medicament. The composition may further comprise an excipient. The skilled person is aware that the excipient may be selected among others based on mode of administration. Suitable excipients can be selected by the skilled person. In an embodiment the excipient may be a biomaterial.

Therefore in an embodiment the composition further comprises a biomaterial. When used herein a biomaterial is a substance that has been engineered to take a form which, alone or as part of a complex system, is used to direct, by control of interactions with components of living systems, the course of any therapeutic or diagnostic procedure, in human or veterinary medicine. Non limiting examples of a biomaterial are:-molecules directly binding to the microRNAs in the composition, such as for example aptamers;-extracellular vesicles;-nanoparticles, such as lipid nanoparticles;
polymers, such as natural polymers or synthetic polymers;-3D scaffold based delivery system such as for example hydrogels. Additional biomaterials are described for example in Williams (2009) Biomaterials, Volume 30, Issue 30, October 2009, Pages 5897-5909, hereby incorporated by reference in its entirety.

A molecule directly binding the microRNA may for example be a targeting moiety, such as a peptide, antibody, antigen binding domain, polysaccharide, tag, nucleotide or aptamer. When used herein an aptamer is a short sequence of artificial DNA or RNA that bind a specific target molecule.

Extracellular vesicles are lipid bilayer-delimited particles that are naturally released from cells. Extracellular vesicles may be used to deliver microRNAs to a specific site or target cell.

A nanoparticle is a small particle usually with a size of about 1 to 100 nanometers. The particle may be a lipid nanoparticle, for example a lipid bilayer enclosing an aqueous core (liposomes), or a lipid layer enclosing a polar core (solid lipid nanoparticle). Alternatively inorganic nanoparticles, polyplexes or dendrimers may be used. Lipid may further comprise polymers or proteins (such as for example apoproteins) to enhance stability, and/or comprise cationic lipids to allow loading of polar compounds (such as microRNAs) in a lipophilic core or attached to a lipid (bi) layer. Nanoparticles can be loaded with microRNA (e.g. in the core or bound to the surface) to deliver the microRNA to a desired site, e.g. an organ, tissue type or cell.

Polymers may be used to deliver microRNAs to a target site. Furthermore polymers could be used as a transfecting agent such as for example polyethylenimine (PEI). Non limiting examples of polymers that may be used as carriers for microRNAs are PDAPEI, polylactic acid (PLA), poly(lactic-co-glycolic acid), dendrimers and chitosan, but the skilled person will be aware of other suitable polymers. Dendrimers are highly ordered, branched polymeric molecules. Non-limiting examples are Polyamidoamine (PAMAM) and Poly(propylene imine) (PPI) based dendrimers.

3D scaffold based delivery system may be used to release microRNAs at a specific site in a subject. An example of a 3D scaffold based delivery system is a hydrogel. The hydrogel is preferably biocompatible. Non limiting examples of biocompatible hydrogels are polyethylenglycol (PEG) based hydrogels, PLGA based hydrogels, cellulose based hydrogels and polyacrylamide (PAM) based hydrogels. Exemplary hydrogels are for example described in Aswathy S H et al. Heliyon. 2020 Apr. 7;6 (4): e03719, hereby incorporated by reference in its entirety.

Thus in an embodiment the composition further comprises a biomaterial selected from a vesicle, a nanoparticle, a polymer or a 3D scaffold based delivery system. In an embodiment the composition further comprises an aptamer, preferably wherein the aptamer is covalently attached to the one or more microRNA.

It is further envisioned that the microRNAs as broadly defined herein above (e.g. one or more, or two or more, etc. microRNAs selected from hsa-miR-4691-5p (guccuccaggccaugagcugcgg), hsa-miR-4496 (gaggaaacugaagcugagaggg), hsa-miR-582-5p (uuacaguuguucaaccaguuacu), and hsa-miR-345-3p (gcccugaacgaggggucuggag) or a variant thereof) can be expressed in a cell using a nucleotide encoding the microRNA. Thus in second aspect the invention relates to a pharmaceutical composition comprising a nucleotide encoding a microRNA as defined in any one of the preceding claims. When used herein the term nucleotide refers to a poly nucleotide which may be RNA or DNA or a combination thereof. When used herein, the term “encoding a microRNA” implies that the microRNA can be expressed, e.g. from the nucleotide. For example, the nucleotide may be an expression vector with a promoter driving the expression of the one or more (e.g. two, three or all four) microRNAs selected from hsa-miR-4691-5p, hsa-miR-4496, hsa-miR-582-5p, and hsa-miR-345-3p (or variants thereof). The nucleotide may also be a viral vector. It is understood that any nucleic acid construct capable of expressing the microRNAs or variants thereof are intended to be covered.

Thus the nucleotide encodes one or more (e.g. two, three or all four) sequences comprising or consisting a sequence as defined in SEQ ID NO: 1, SEQ ID NO: 2, SEQ ID NO: 3 and/or SEQ ID NO: 4 or variants thereof as defined herein above. Thus the nucleotide comprises a sequence as defined in SEQ ID NO: 1, SEQ ID NO: 2, SEQ ID NO: 3 and/or SEQ ID NO: 4 or variants thereof or comprises a sequence reverse complement to a sequence as defined in SEQ ID NO: 1, SEQ ID NO: 2, SEQ ID NO: 3 and/or SEQ ID NO: 4 or variants thereof. Alternatively the nucleotide encodes one or more (e.g. two, three or all four) sequences comprising or consisting a sequence as defined in SEQ ID NO: 5, SEQ ID NO: 6, SEQ ID NO: 7 and/or SEQ ID NO: 8 or variants thereof as defined herein above. Thus the nucleotide comprises a sequence as defined in SEQ ID NO: 5, SEQ ID NO: 6, SEQ ID NO: 7 and/or SEQ ID NO: 8 or variants thereof or comprises a sequence reverse complement to a sequence as defined in SEQ ID NO: 5, SEQ ID NO: 6, SEQ ID NO: 7 and/or SEQ ID NO: 8 or variants thereof.

In an embodiment the nucleotide encodes two or more microRNAs (e.g two, three or all four) selected from hsa-miR-4691-5p (guccuccaggccaugagcugcgg), hsa-miR-4496 (gaggaaacugaagcugagaggg), hsa-miR-582-5p (uuacaguuguucaaccaguuacu), and hsa-miR-345-3p (gcccugaacgaggggucuggag) or variants thereof. For example the two or more microRNAs or variants thereof are a combination of two, three or four microRNAs as provided above.

The microRNAs as broadly defined herein may also be delivered using a viral particle. The viral particle may for example comprise the microRNA, or encode the microRNA. Thus in a third aspect the invention relates to a viral particle comprising the microRNA as defined in the first aspect of the invention or a nucleotide encoding a microRNA as defined in the first aspect of the invention. Suitable viral vectors that could be used are for example adeno-associated-virus (AAV) vectors, adenovirus vectors, or lentivirus vectors. Exemplary methods for viral vector based gene therapy are for example reviewed in Bulcha, J. T. et al. Sig Transduct Target Ther 6, 53 (2021), hereby incorporated by reference in its entirety. The viral particle may be comprised in a pharmaceutical composition as defined herein above and may thus further comprise a biomaterial such as a vesicle, a nanoparticle, a polymer a 3D scaffold based delivery system or an aptamer.

In a further aspect it is envisioned that the compositions, microRNAs, nucleotides and viral particles are used as a medicament. Thus in an aspect the invention relates to a microRNA as defined in the first aspect of the invention or the nucleotide as defined in the second aspect of the invention or the viral particle according to the third aspect of the invention for use as a medicament. Particularly the invention relates to a microRNA as defined in the first aspect of the invention or the nucleotide as defined in the second aspect of the invention or the viral particle according to the third aspect of the invention for use in the treatment, prevention or amelioration of a disease. Preferably the use comprises inducing or stimulating neovascularization.

Alternatively, the invention relates to a method of treatment, the method comprising administering a microRNA as defined in the first aspect of the invention or the nucleotide as defined in the second aspect of the invention or the viral particle according to the third aspect of the invention to a subject in need thereof. Further the invention relates to a method of in the treating, preventing or ameliorating a disease the method comprising administering a microRNA as defined in the first aspect of the invention or the nucleotide as defined in the second aspect of the invention or the viral particle according to the third aspect of the invention to a subject in need thereof. Preferably the method comprises inducing or stimulating neovascularization.

It is further envisioned that a therapy is based on the use of two or more (e.g. two, three or all four) microRNAs, or a nucleotide encoding two or more microRNAs or a viral particle comprising or encoding two or more microRNAs as broadly defined herein. Thus the use (or method of treatment) envisions combinations of microRNAs as broadly described herein above.

As the microRNAs described herein have a proangiogenic effect their use is envisaged in coronary heart disease, myocardial infarction or stroke. In an embodiment the use comprises inducing neovascularization. For example an application is envisioned to stimulate neovascularization after myocardial infarction to restore or partially restore blood flow to the myocardium, by locally administering the microRNA, nucleotide or viral particle as broadly described herein. Further envisioned is for example the use of the microRNA, nucleotide or viral particle as broadly described herein to stimulate neovascularization after cerebral stroke.

### Examples

Having now generally described the invention, the same will be more readily understood through reference to the following examples which is provided by way of illustration and is not intended to be limiting of the present invention.

### Results

a Systematic Review of the Literature and an in-Silico Analysis Identified 6 Novel miRNAs with a Potential Role in Angiogenesis

To identify novel miRNAs with a possible role in angiogenesis we first performed a systematic review of the literature to enlist all miRNAs with previously reported roles in the regulation of angiogenesis regardless of the field of study. After screening 4,312 publications and extracting data from 284 eligible studies we identified 93 angiogenic and 192 anti-angiogenic miRNAs with experimentally validated roles in angiogenesis. To expand the list of miRNAs with a possible role in angiogenesis we performed an unbiased in-silico screen to find novel miRNAs predicted to involve in angiogenesis pathways. Using miRPathDBv2.02 we identified 39 novel miRNAs predicted to involve in the VEGF angiogenesis pathway by both DIANA-microT and miRDB and Target Scan target prediction tools (p<0.001). The possible implication of these miRNAs in cell survival pathways was assessed using miRPathDB v2.02. A total of 14 miRNAs that were not predicted to involve in cell survival pathways and were selected for further investigation. For these miRNA candidates, further information was collected, including conservation, association with EVs, tissue and cell expression. Finally, 6 miRNA candidates were selected for experimental validation (table 1).

TABLE 1
Information on the 6 novel miRNA candidates with a possible role in angiogenesis. Information
on conservation was extracted by miRNAminer. Tissue expression was investigated using
the miRNA Tissue Atlas, miRGator and mESAdb. The presence of the 6 miRNA candidates
in EC datasets was examined by mining publicly available datasets on ENCODE. A prediction
of the top targets involved in the VEGF pathway was performed using miRPathDB and
the R package multimiR and the presence of the 6 novel miRNA candidates in EVs was
evaluated by mining the EV-miRNA repositories Vesiclepedia and Exocarta.

| | | | Presence in | | |
|---|---|---|---|---|---|
| | | | EC datasets | | |
| | | | (PGP1-EC, | | |
| | | | hSaVECs, | Top Predicted | |
| | | Tissue | thoracic aorta | targets (VEGF/ | Presence |
| miRNA | Conservation | Expression | ECs, HUVECs) | PDGF path) | in EVs |
| hsa-miR- | high (hsa, | Expressed in | EMERGED IN | CRK, GPC1, | Yes |
| 345-3p | mmu, rno | many tissues | EC DATASETS | ITCH, | (Found in |
| | etc.) | (including arteries, | (hSaVECs, | MKNK1, | mouse and |
| | | myocardium) | thoracic aorta | PAK2, | rat EVs) |
| | | | ECs, HUVECs) | PRKCI | |
| hsa-miR- | low (hsa, | — | EMERGED IN | CTNNA1, | No |
| 579-3p | ptr, ppy, | | EC DATASETS | EEA1, | |
| | mml etc.) | | (PGP1-ECs, | FOXO3, | |
| | | | hSaVECs, | JAG1, | |
| | | | thoracic aorta | PIK3R1, | |
| | | | ECs) | PRKCB, | |
| | | | | RAB11A, | |
| | | | | RAP1A, | |
| | | | | RPS6KB1, | |
| | | | | STAM | |
| hsa-miR- | high (hsa, | Low | EMERGED IN | BMP10, | Yes |
| 582-5p | mmu, rno | expression in | EC DATASETS | EIF4E, | (Found in |
| | etc.) | arteries, no | (hSaVECs, | FOXO1, | human and |
| | | expression in | thoracic aorta | GSK3B, | mouse EVs) |
| | | the myocardium | ECs, HUVECs) | MAPK14, | |
| | | | | RICTOR | |
| hsa-miR- | low (hsa, | Expressed in | NOT | CRK, | No |
| 3692-3p | ggo, ppy, | several tissues | EMERGED IN | MEF2C, | |
| | mml etc.) | (including the | EC DATASETS | PRKCB, | |
| | | myocardium) | (in any of the 4 | PTPRZ1 | |
| | | | EC datasets) | | |
| hsa-miR- | — | Expressed in | NOT | CTNNB1, | No |
| 4496 | | many tissues | EMERGED IN | HDAC4, | |
| | | (including | EC DATASETS | HDAC9, | |
| | | arteries, | (in any of the 4 | KL, | |
| | | myocardium) | EC datasets) | NCK1, | |
| | | | | RAP1B, | |
| | | | | ROCK2 | |
| hsa-miR- | — | Low | NOT | EIF2AK3, | No |
| 4691-5p | | expression in | EMERGED IN | PRKCB | |
| | | arteries, no | EC DATASETS | | |
| | | expression in | (in any of the 4 | | |
| | | the myocardium | EC datasets) | | |

Expression levels of the six miRNA candidates was assessed by real-time quantitative PCR (qPCR) in different types of endothelial and smooth muscle cells. Our results showed negligible expression of the miRNAs miR-579-3p, miR-4496, miR-345-3p, miR-3692-3p, miR-4691-5p in endothelial and smooth muscle cell populations. Moreover, we showed low expression of mir-582-5p in HCMECs (FIG. 1).

MiRNA-345-3p, miRNA-582-5p, miRNA-4496 and miRNA-4691-5p Induce HCMEC Tube Formation and Migration

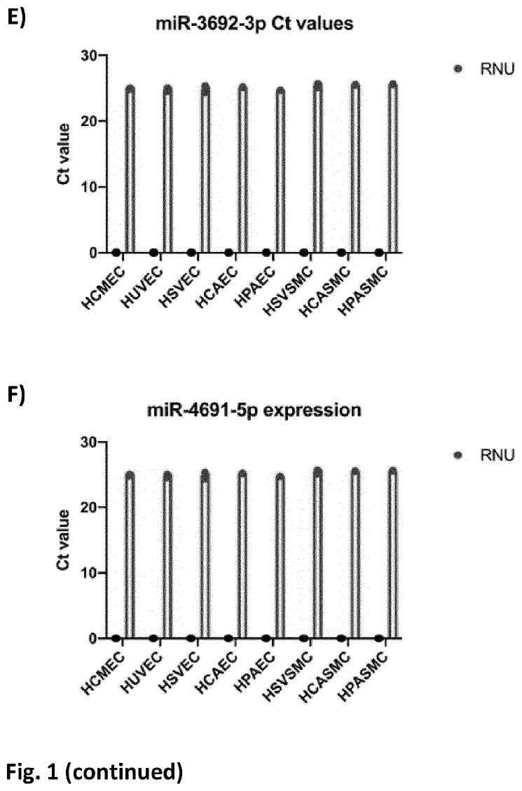

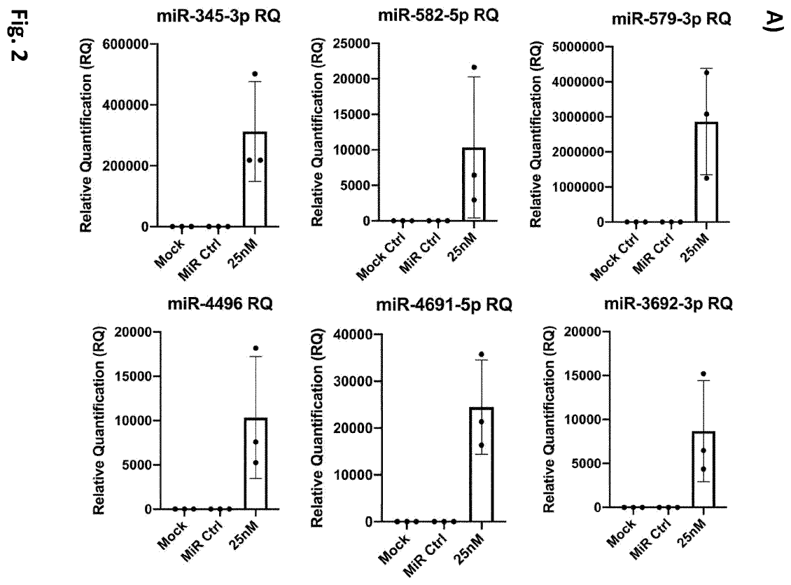

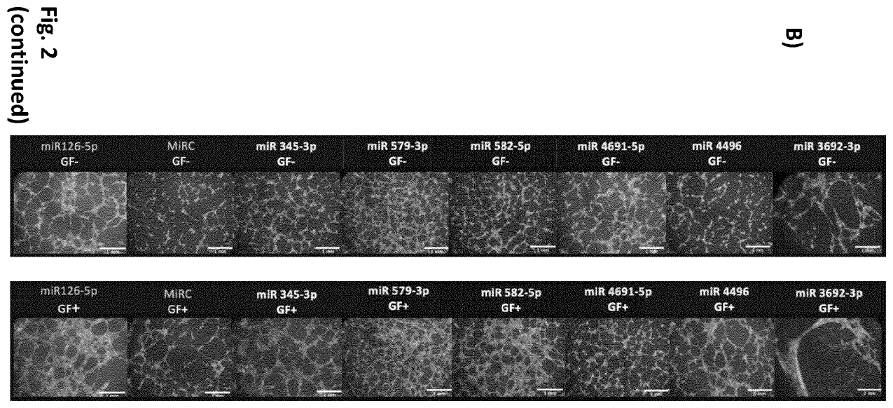

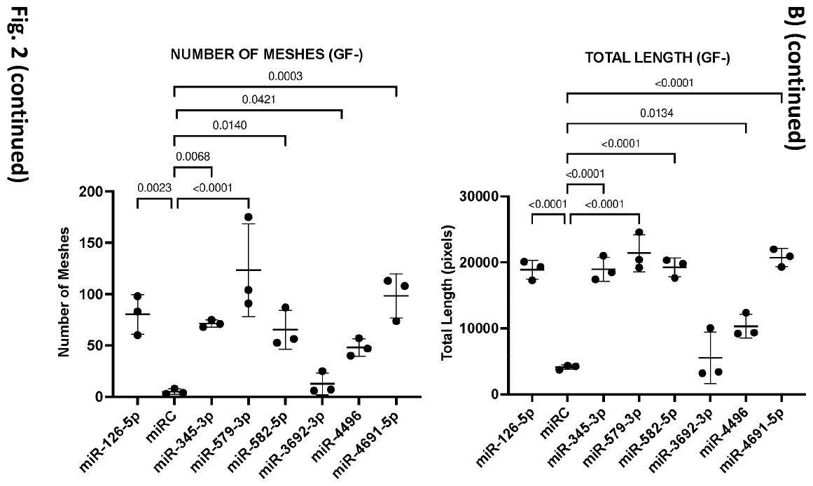

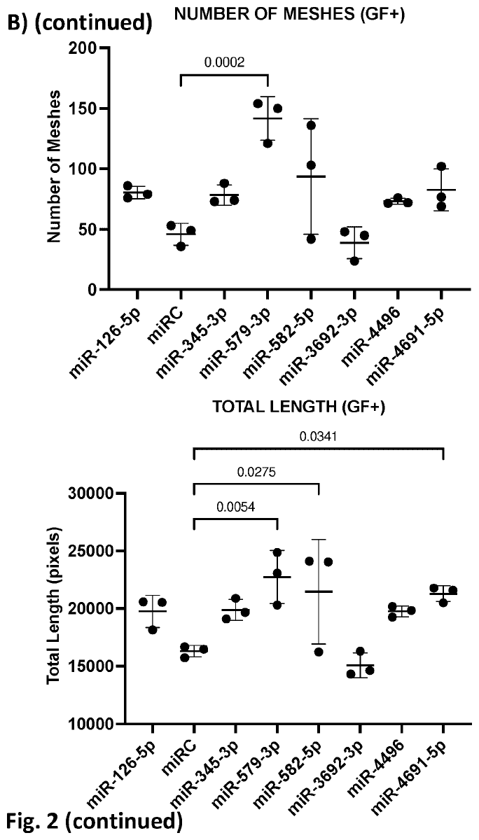

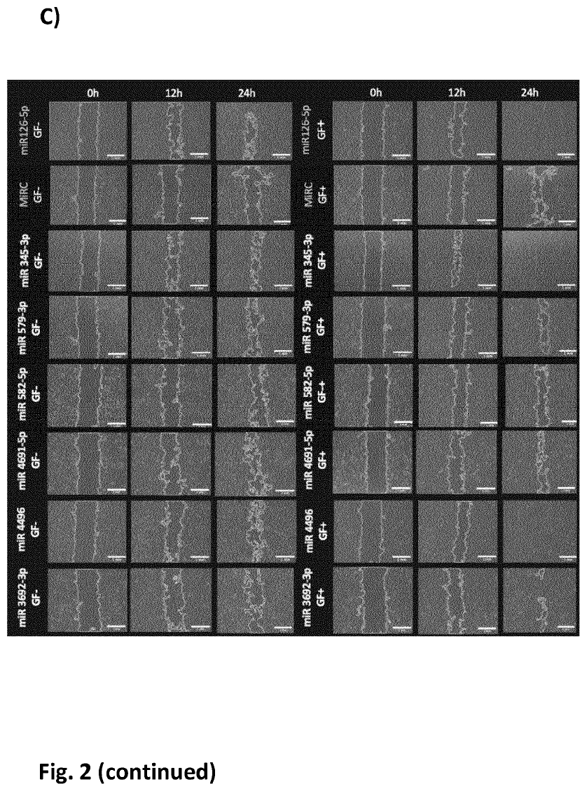

Increased EC tube formation, migration and proliferation are considered hallmarks of angiogenesis³, thus to assess the angiogenic potency of the 6 novel miRNAs with potential roles in angiogenesis we performed tube formation, migration and proliferation assays in human cardiac microvascular endothelial cells (HCMECs) overexpressing the 6 miRNA candidates. Since the role of miR-126-5p in angiogenesis is extensively studied 4-7, HCMECs overexpressing miR-126-5p were used as positive controls of angiogenic/migratory cells. HCMECs transfected with a miRNA control (miRC) were used as negative controls. MiRNA overexpression was confirmed by qPCR (FIG. 2A). Since our in-silico analysis revealed that the candidate miRNAs are involved in the VEGF angiogenesis pathway but their role in angiogenesis was unknown, experiments were performed both in basal (GF−) and in fully supplemented (GF+) media to identify miRNAs with an angiogenic or an anti-angiogenic action, respectively. Using tube formation assay, we showed that all the candidate miRNAs apart from hsa-miR-3692-3p significantly increased the number of meshes and total length of the tubes formed compared to the miRC when cells were cultured in basal media (p<0.05) suggesting that these are potentially angiogenic miRNAs (FIG. 2B). To assess the effect of the 6 candidate miRNAs on HCMEC migration, we performed scratch assays (FIG. 2C). We demonstrated that 24 h after the induction of the wound, HCMECs transfected with all 6 novel miRNAs and cultured in basal media had significantly improved migratory ability compared to the miRC (p<0.001). Finally, we sought to investigate the effect of miRNA overexpression in HCMEC proliferation. Our data showed that when HCMECs were cultured under basal conditions miR-582-5p and miR-4496 overexpression significantly improved HCMEC proliferation ability (p=0.0124 and p=0.0180 respectively), while miR-579-3p and miR-3692-3p had an anti-proliferative effect (p=0.0018 and p=0.0126 respectively) (FIG. 2D). MIR-4691-5p overexpression resulted in improved HCMEC proliferation ability only when cells were cultured in fully supplemented media (p=0.0009), while miRNA-345-3p did not affect EC proliferation in any condition studied. Collectively, our data highlight miR-345-3p, miR-582-5p, miR-4496 and miR-4691-5p as novel angiogenic miRNAs.

Transcriptomic Analysis Revealed the Regulation of Cell Cycle Genes Following Overexpression of all the 4 Novel Angiogenic miRNAs in HCMECs.

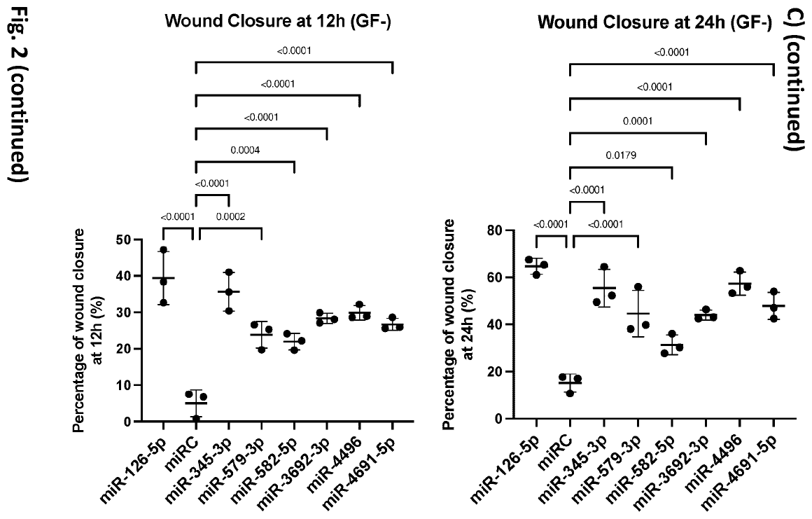

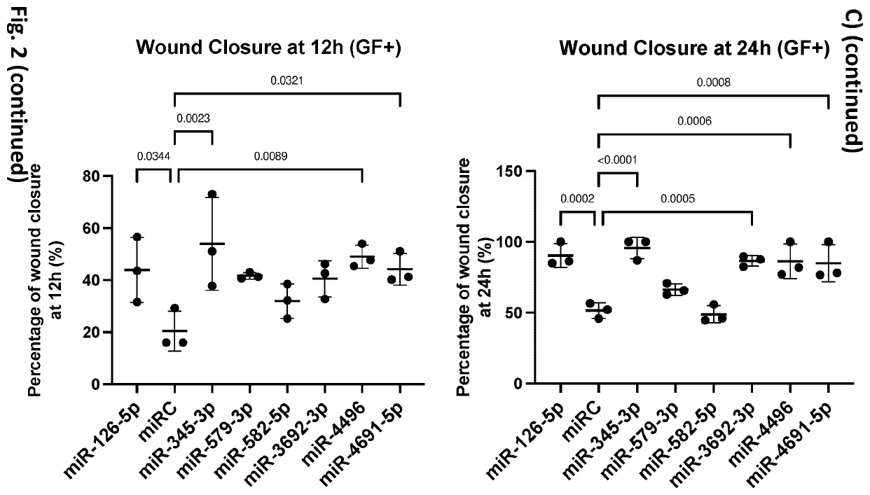

To understand the transcriptomic changes associated with miR-345-3p, miR-582-5p, miR-4496 and miR-4691-5p overexpression in HCMECs we performed deep RNA sequencing. Following RNA sequencing, principal component analysis (PCA) confirmed clustering of the sample replicates and showed a clear separation of samples overexpressing the different miRNAs compared to miRC-transfected samples (FIG. 3). We also noted that samples overexpressing miR-582-5p or miR-4691-5p had distinct transcriptomic profiles, while samples overexpressing miR-345-3p or miR-4496 clustered closer implying the regulation of a similar set of genes.

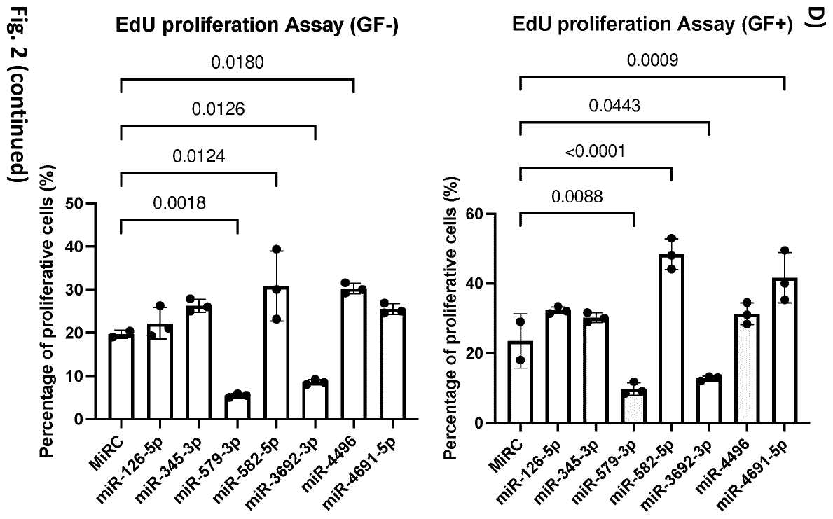

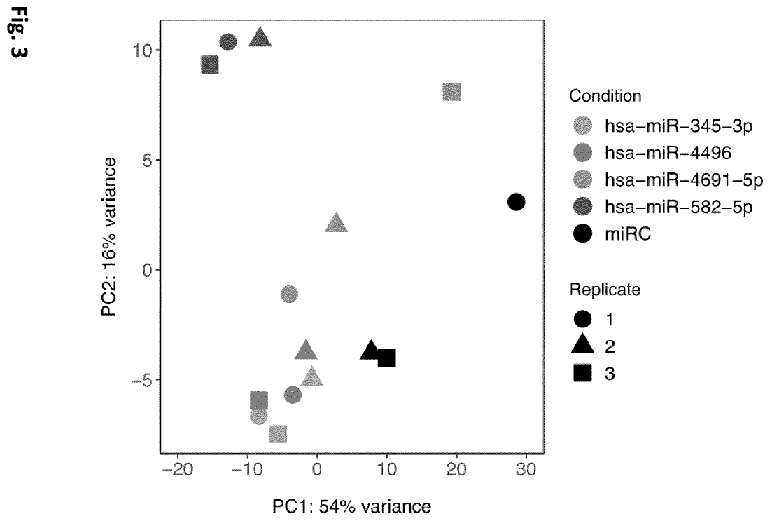

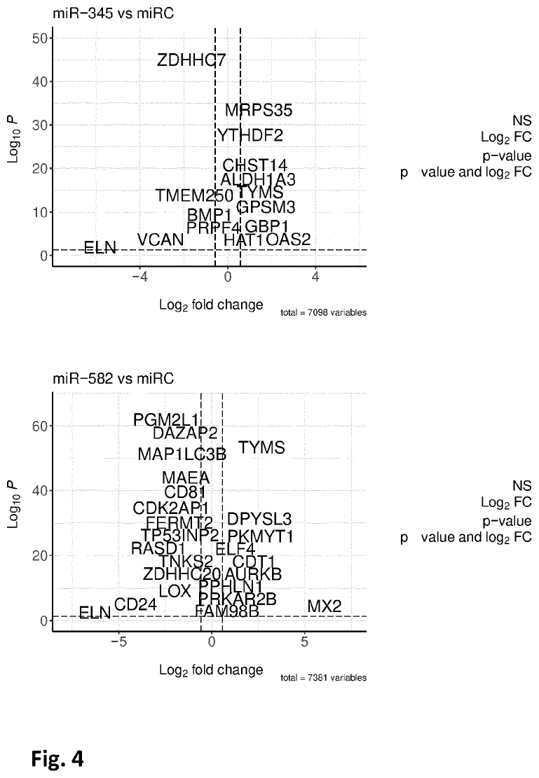

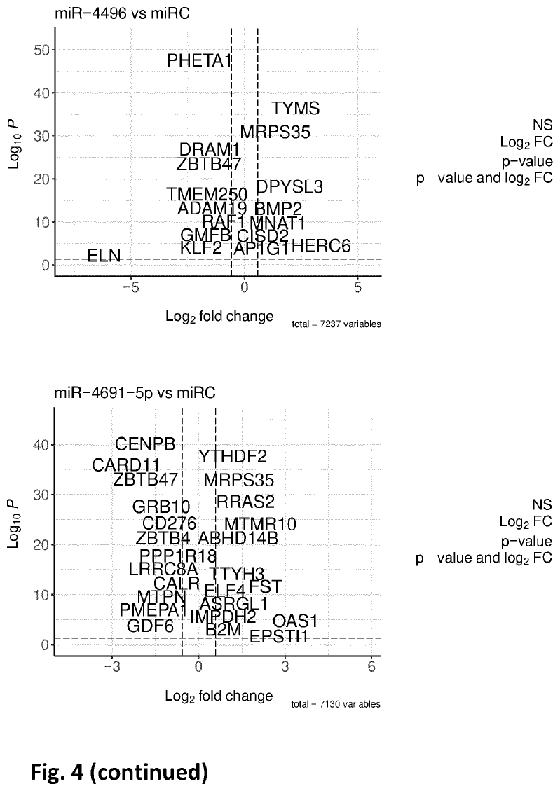

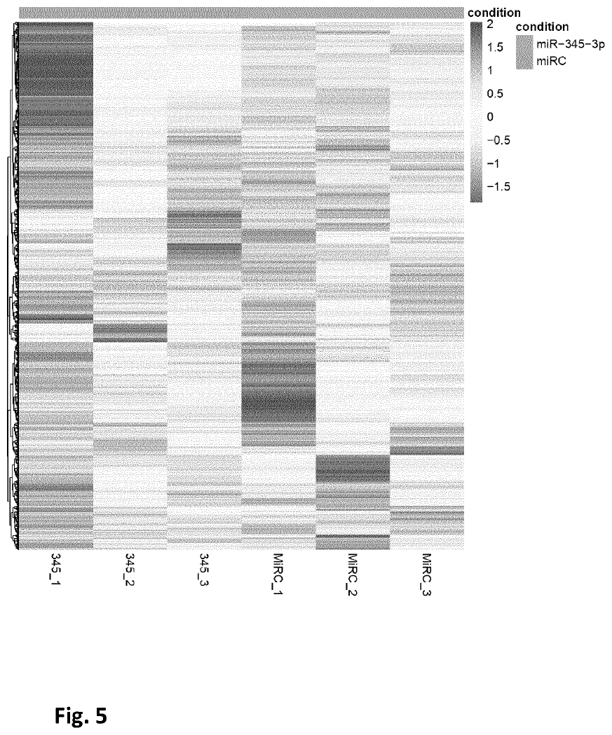

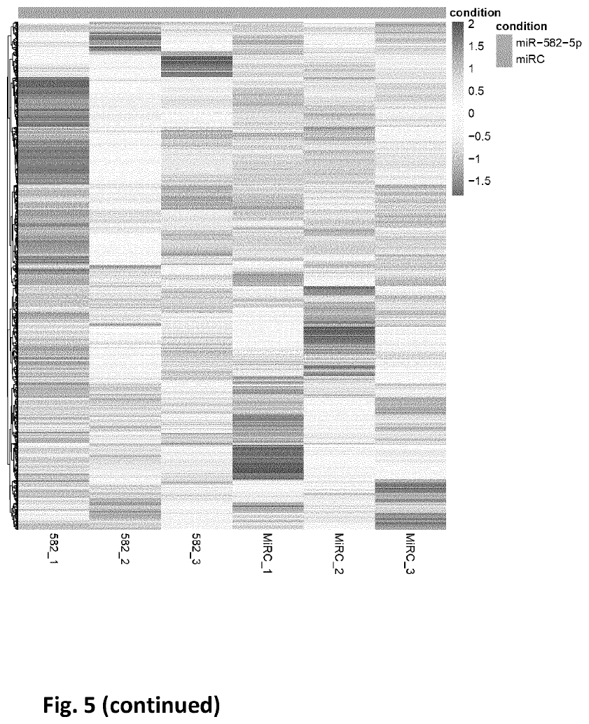

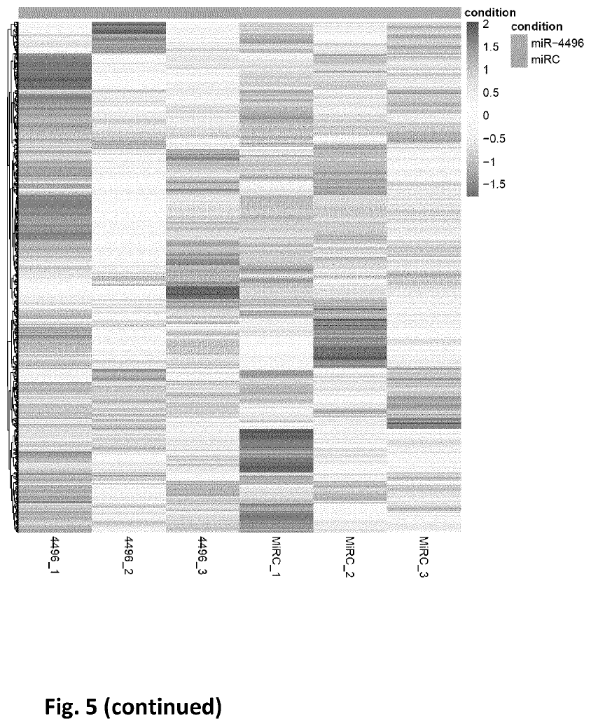

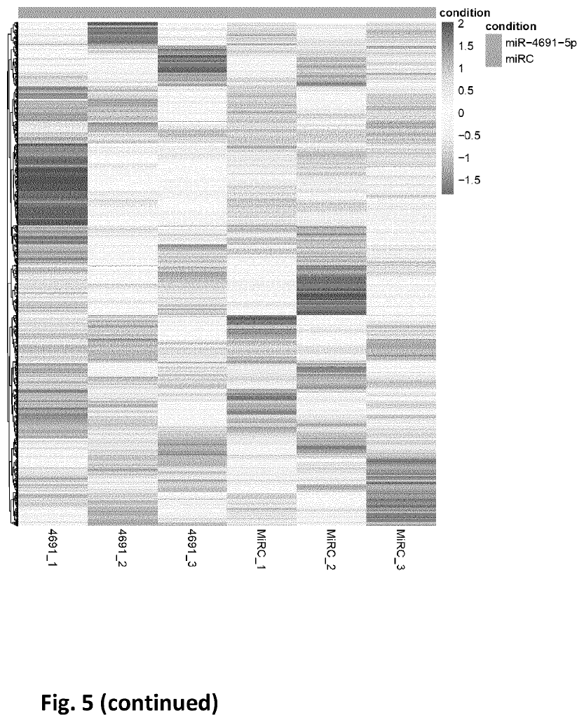

We then sought to analyse the significant changes in gene expression in the 4 novel angiogenic miRNA overexpressing HCMECs, compared to the miRC transfected samples. By performing differential expression analysis, we found that miRNA overexpression affected a large number of genes compared to the miRC-transfected samples (FIG. 4). In total, we found 534 genes being upregulated post-miR-345-3p overexpression, while 254 genes were downregulated (FIG. 5). MIR-582-5p resulted in the upregulation of 905 genes and downregulation of 892 genes. A total of 557 genes were upregulated post-miR-4496 overexpression, while 468 genes were downregulated. Finally, overexpression of miR-4691-5p resulted in the upregulation of 386 genes, and the downregulation of 422 genes.

### Key Points

Overall, we identified 4 novel miRNAs which effectively induced HCMEC angiogenesis, possibly by the regulation of cell cycle-associated genes. By qRT-PCR we showed that the 4 novel angiogenic miRNAs are absent in most ECs tested. Finally, by mining EV-miRNA repositories we found that 2 of these miRNAs (miR-345-3p and miR-582-5p) have been previously identified in mouse EV-RNA sequencing datasets. In our preliminary EV-small RNA sequencing data we have demonstrated that miR-4496 and miR-4691-5p can also be transferred through EVs. The sorting of these miRNAs into EVs may be facilitated due to the presence of specific motifs in their sequence. MIR-345-3p contains 2 EV-sorting motifs previously published by Garcia-Martin et al. 8:1) GGAG and 2) GAGGG, miR-4496 sequence contains the motif GAGGG, while miR-4691-5p contains the motifs: 1) CAUG and 2) AGGCC. Although several miRNAs have been shown to increase angiogenesis to date9, miR-345-3p, miR-582-5p, miR-4496, miR-4691-5p appear as very promising candidates for therapeutic angiogenesis due to 2 main reasons: 1) their absence/low expression in ECs favours overexpression-related therapeutic strategies, 2) their ability to get sorted into EVs broadens the range of therapeutic strategies that may be exploited to target these miRNAs to ECs.

### REFERENCES

1. Moher, D., Liberati, A., Tetzlaff, J. & Altman, D. G. Preferred Reporting Items for Systematic Reviews and Meta-Analyses: The PRISMA Statement. PLoS Med 6, e1000097 (2009).
2. Kehl, T. et al. MiRPathDB 2.0: A novel release of the miRNA Pathway Dictionary Database. Nucleic Acids Res 48, D142-D147 (2020).
3. Guo, S. et al. Assays to examine endothelial cell migration, tube formation, and gene expression profiles. Methods Mol Biol 1135, 393-402 (2014).
4. Qu, M. et al. MicroRNA-126 Regulates Angiogenesis and Neurogenesis in a Mouse Model of Focal Cerebral Ischemia. Mol Ther Nucleic Acids (2019) doi: 10.1016/j.omtn.2019.02.002.
5. Wang, S. et al. The Endothelial-Specific MicroRNA miR-126 Governs Vascular Integrity and Angiogenesis. Dev Cell 15, 261-271 (2008).
6. Fish, J. E. et al. miR-126 Regulates Angiogenic Signaling and Vascular Integrity. Dev Cell 15, 272-284 (2008).
7. Jakob, P. et al. Loss of angiomir-126 and 130a in angiogenic early outgrowth cells from patients with chronic heart failure: Role for impaired in vivo neovascularization and cardiac repair capacity. Circulation 126, 2962-2975 (2012).
8. Garcia-Martin, R. et al. MicroRNA sequence codes for small extracellular vesicle release and cellular retention. Nature 2021 601:7893 601, 446-451 (2021).
9. Kesidou, D. et al. Extracellular Vesicle miRNAs in the Promotion of Cardiac Neovascularisation. Front Physiol 11, (2020).

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding patent applications, patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Sequences used in the application:

SEQ ID NO: 2
gaggaaactg aagctgagag gg
SEQ ID NO: 3
ttacagttgt tcaaccagtt act
SEQ ID NO: 4
gccctgaacg aggggtctgg ag
SEQ ID NO: 5
ggagcactcc caggtcctcc aggccatgag
ctgcggccct gatgtctcta ctccagccac
ggactgagag tgcataggag tgtcc
SEQ ID NO: 6
acatcagctc atataatcct cgaagctgcc
tttagaaatg aggaaactga agctgagagg
g
SEQ ID NO: 7
atctgtgctc tttgattaca gttgttcaac
cagttactaa tctaactaat tgtaactggt
tgaacaactg aacccaaagg gtgcaaagta
gaaacatt
SEQ ID NO: 8
acccaaaccc taggtctgct gactcctagt
ccagggctcg tgatggctgg tgggccctga
acgaggggtc tggaggcctg ggtttgaata
tcgacagc

1. A method of treating, preventing or ameliorating a cardiovascular disease in a subject in need thereof by inducing neovascularization, comprising administering a composition for inducing neovascularization to the subject
wherein the composition comprises one or more microRNAs selected from the group consisting of hsa-miR-4691-5p (guccuccaggccaugagcugcgg, SEQ ID NO: 1), hsa-miR-4496 (gaggaaacugaagcugagaggg, SEQ ID NO: 2), hsa-miR-582-5p (uuacaguuguucaaccaguuacu, SEQ ID NO: 3), and hsa-miR-345-3p (gcccugaacgaggggucuggag, SEQ ID NO: 4) or variants thereof.

2. The method of claim 1 wherein the variant comprises or consist of a nucleotide sequence with a sequence identity of at least 70% to hsa-miR-4691-5p, hsa-miR-4496, hsa-miR-582-5p, and hsa-miR-345-3p, or wherein said variant comprises or consists of a nucleotide sequence containing up to 8 altered nucleotides in hsa-miR-4691-5p, hsa-miR-4496, hsa-miR-582-5p, and hsa-miR-345-3p.

3. The method of claim 1 wherein the variant comprises or consist of a nucleotide sequence with a sequence identity of at least 70% to SEQ ID NO: 1, SEQ ID NO: 2, SEQ ID NO: 3, and SEQ ID NO: 4, or wherein said variant comprises or consists of a nucleotide sequence containing up to 8 altered nucleotides in SEQ ID NO: 1, SEQ ID NO: 2, SEQ ID NO: 3, and SEQ ID NO: 4.

4. The method of claim 1 wherein the one or more microRNA has a sequence with at least 70% sequence identity with SEQ ID NO: 5, SEQ ID NO:6, SEQ ID NO: 7 or SEQ ID NO: 8.

5. The method of claim 1 wherein the microRNA has a pro-angiogenic effect when expressed in an endothelial cell.

6. The method of claim 1, wherein the composition comprises two or more, preferably three, more preferably all four, microRNAs selected from the group consisting of hsa-miR-4691-5p, hsa-miR-4496, hsa-miR-582-5p, and hsa-miR-345-3p or variants thereof.

7. The method of claim 1 wherein the composition further comprises a biomaterial.

8. The method of claim 7 wherein the biomaterial is selected from a vesicle, a nanoparticle, a polymer or a 3D scaffold based delivery system.

9. The method of claim 1, wherein the composition further comprises an aptamer, preferably wherein the aptamer is covalently attached to the one or more microRNA.

10. The method of claim 1, wherein the composition is a pharmaceutical composition.

11. The method of claim 1, wherein the composition comprises a viral particle comprising the microRNA as defined in any one of claims 1-9 or a nucleotide encoding a microRNA as defined in any one of claims 1-9.

12. The method of claim 1 wherein the cardiovascular disease is coronary heart disease, myocardial infarction or stroke.


SEQUENCE LISTING (8 sequences)

SEQ ID NO: 1 - RNA, 23 nt, Homo sapiens
  source 1..23: mol_type = other RNA
gtcctccaggccatgagctgcgg

SEQ ID NO: 2 - RNA, 22 nt, Homo sapiens
  source 1..22: mol_type = other RNA
gaggaaactgaagctgagaggg

SEQ ID NO: 3 - RNA, 23 nt, Homo sapiens
  source 1..23: mol_type = other RNA
ttacagttgttcaaccagttact

SEQ ID NO: 4 - RNA, 22 nt, Homo sapiens
  source 1..22: mol_type = other RNA
gccctgaacgaggggtctggag

SEQ ID NO: 5 - RNA, 85 nt, Homo sapiens
  source 1..85: mol_type = other RNA
ggagcactcccaggtcctccaggccatgagctgcggccctgatgtctctactccagccacggactgagagtgcataggagtgtcc

SEQ ID NO: 6 - RNA, 61 nt, Homo sapiens
  source 1..61: mol_type = other RNA
acatcagctcatataatcctcgaagctgcctttagaaatgaggaaactgaagctgagaggg

SEQ ID NO: 7 - RNA, 98 nt, Homo sapiens
  source 1..98: mol_type = other RNA
atctgtgctctttgattacagttgttcaaccagttactaatctaactaattgtaactggttgaacaactgaacccaaagggtgcaaagtagaaacatt

SEQ ID NO: 8 - RNA, 98 nt, Homo sapiens
  source 1..98: mol_type = other RNA
acccaaaccctaggtctgctgactcctagtccagggctcgtgatggctggtgggccctgaacgaggggtctggaggcctgggtttgaatatcgacagc